(19) United States
(12) Patent Application Publication
(10) Pub. No.: US 2024/0426804 A1
(43) Pub. Date: Dec. 26, 2024

(54) Systems and Methods of Soil Composition Analysis Using Combined Soil Samples

(71) Applicant: EarthOptics, Inc., Arlington, VA (US)

(72) Inventor: Lars Dyrud, Great Falls, VA (US)

(73) Assignee: EarthOptics, Inc., Arlington, VA (US)

(21) Appl. No.: 18/755,258

(22) Filed: Jun. 26, 2024

Related U.S. Application Data
(60) Provisional application No. 63/510,170, filed on Jun. 26, 2023.

(51) Int. Cl. G01N 33/24; G01N 21/17

(52) U.S. Cl. CPC G01N 33/24; G01N 21/17; G01N 2021/1787

(57) ABSTRACT

A method for soil composition analysis includes collecting initial soil samples from cells of a soil area; homogenizing the collected initial soil samples; combining the homogenized initial soil samples from at least two cells to produce combined soil samples such that each cell is represented in at least two separate combined samples; analyzing soil composition of the combined samples; and performing mathematical inversion (for example, tomographic) to determine soil composition of the initial soil samples and/or the corresponding cells. The method can further include using the composition of the original samples to determine composition of the adjacent soil areas. The samples can be collected over a soil area represented as a 2D or 3D grid (wherein the cells of the grid can be of uniform or nonuniform size and/or shape). Analyzing the composition of the soil can include physical, chemical and/or biological aspects of the soil area.

### FIELD OF THE INVENTION

The present disclosure relates to agronomy and agriculture, and more particularly to methods of soil composition analysis.

### BACKGROUND

Unless otherwise indicated herein, the materials described herein are not prior art to the claims in the present application and are not admitted to be prior art by inclusion in this section.

Generally speaking, modern agriculture and agronomy heavily rely on soil composition analysis. Typically, such an analysis requires soil samples to be collected over multiple locations in a given field, followed by a costly analysis carried out in a laboratory. Some attempts have been made to simulate soil characteristics based on the collected samples, but the required number of such samples remains high. For example, in the U.S. Pat. No. 10,275,550 issued to Lee, agricultural intelligence computer system stores a digital model of nutrient content in soil. In the U.S. App. No. 20210010993 by Shibata et al., machine learning and/or artificial intelligence classifier tools are used to process information collected from a large sample set to generate various soil-related output data. In the U.S. App. No. 20220128481 by Islam, methods of measuring the quantity of active organic matter in a soil sample are disclosed. In the U.S. App. No. 20220124961 by Eda et al., an approach to the prediction of soil density and subsoil crop growth is disclosed, and includes using a machine learning model for predicting the soil density around a subsoil crop and the yield of the subsoil crop. The U.S. App. No. 20220124963 by Taylor et al., discloses systems, methods, and apparatuses for implementing automated data modeling and scaling of a Soil Health Data Fabric. However, neither of the aforementioned approaches offer a way of reducing the number of samples in the first place by means of mixing soil samples and using mathematical inversion to determine the soil composition of the samples and the surrounding areas.

Therefore, a need exists for a method and system for soil composition analysis capable of significantly reducing the number of soil samples necessary for a proper representation of the soil characteristics for a given area.

### SUMMARY

Accordingly, the present invention embraces methods and systems for processing soil samples and analyzing soil composition.

In an exemplary embodiment, a method for soil composition analysis includes defining a segmentation of a soil area into one or more cells; collecting one or more initial soil samples from a plurality of the cells of the soil area; homogenizing the initial soil samples; combining portions of the homogenized soil samples from at least two cells to produce a plurality of combined soil samples; analyzing soil composition of the combined soil samples; creating a geometry matrix using the analyzed soil composition of the combined soil samples; and performing mathematical inversion to determine soil composition of the initial and/or homogenized soil samples, and/or the corresponding cells; wherein the combining portions of the homogenized initial soil samples comprises using a portion of at least one homogenized soil sample from one cell in two or more independent combined soil samples.

In another exemplary embodiment, a method for processing soil samples includes gathering one or more samples of soil from one or more soil cells; mixing the gathered original samples of soil across the one or more cells according to a predetermined algorithm to produce a plurality of mixed samples, wherein each original sample is represented in at least two distinct mixed samples; determining composition of the mixed samples; and applying mathematical inversion to the determined composition of the mixed samples to determine composition of the one or more soil cells.

In an exemplary embodiment, a system for soil composition analysis includes a means for gathering one or more original samples of soil from one or more soil cells; a means for mixing the gathered original samples of soil according to a predetermined algorithm; a means for determining composition of the mixed samples; and a processor unit configured to apply mathematical inversion to the determined composition of the mixed samples to determine composition of the original samples.

The foregoing illustrative summary, as well as other exemplary objectives and/or advantages of various embodiments, and the manner in which the same are accomplished, are further explained within the following detailed description and its accompanying drawings.

### BRIEF DESCRIPTION OF THE DRAWINGS

FIG. 1 schematically depicts a method for soil composition analysis, according to various embodiments.

FIG. 2 schematically depicts a method for processing soil samples, according to various embodiments.

FIG. 3 schematically depicts a block diagram of a system for soil composition analysis, according to various embodiments.

FIG. 4 is a representation of a segmented field as used in an example.

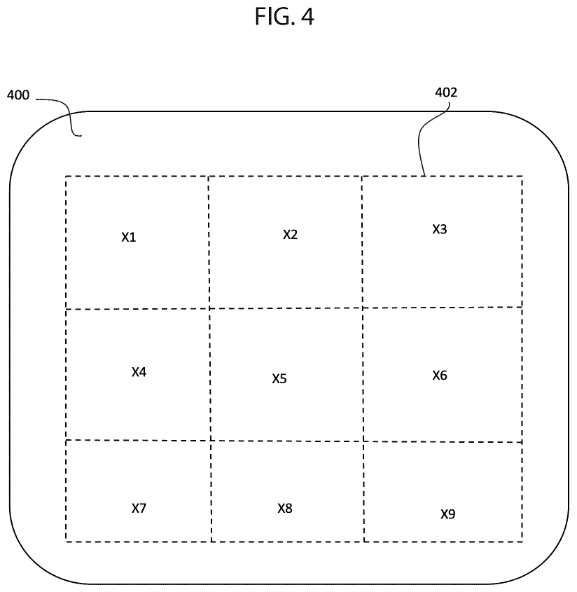

FIG. 5 is a representation of a geometry matrix of a N×M (36×18) two dimensional matrix where values of 1 are represented by black squares.

### DETAILED DESCRIPTION

This application claims priority to U.S. Provisional Application 63/510,170, entitled Soil Composition Analysis with Mathematical Inversion, filed Jun. 26, 2023, the disclosure of which is hereby incorporated by reference.

The present invention embraces various methods and systems for processing soil samples and analyzing soil composition.

Traditional methods for soil sampling are time-consuming and costly, as they require a large amount of samples to be collected and analyzed. The present invention described herein proposes an approach to reduce the necessary number of samples, thus saving time and resources and increasing the efficiency of soil sampling.

As further described below, various methods include sampling of soil at multiple locations and combining the soil samples into combination samples that are tested for soil composition. Multiple combinations soil samples may be prepared, and each soil combination sample may be tested for soil composition. The soil composition test results from the multiple combination soil samples can then be used to determine an estimated or predicted value of the soil composition of each of the original soil samples, without separately testing any of the original soil samples to determine their compositions. The number of combination samples tested may be less than the number of original soil samples used to make the combination samples, thus reducing the number of tests performed while still determining the soil composition at each original soil sample location. Depending on the sampling plan, a substantial cost reduction can be achieved. For example, in the simplest version of a 3×3 matrix, a 33% sample cost reduction can be achieved. A 5×5 matrix can result in a 10/25=60% reduction. A 10×10 can have a 20/100=80% reduction, and so forth.

A large number of samples may be collected and combined to determine estimated values for one or more soil composition variables of interest at each sample location, such as across an entire field. Alternatively, a smaller number of samples may be combined to determine estimated values at the sample locations within a smaller area, such as a portion of a field. This process of determining soil composition at locations in a smaller area such as a portion of a field may be repeated across multiple contiguous portions of the field such that, when the results are combined, estimated values may be determined across the larger area, such as the entire field or portions of the field included in the combined smaller areas. For example, samples could be obtained from a 10 by 10 grid of 100 sample locations across a large field. These samples could all be combined to form 20 combination samples each containing soil from 10 different sample locations. Alternatively, the same large field of 100 sample locations could be divided into four smaller areas, each including a 5 by 5 grid of sample locations with 25 samples obtained from each quarter of the field. Ten combination samples, each sample from 5 different sample locations, could be tested for each quarter of the field. The determined results for the 25 sample locations of each of the four smaller areas could be combined to determine results for the entire field of 100 sample locations. In both cases, 100 samples are collected. In the first case, 20 combination samples are tested as compared to 40 combination samples in the second case. However, in both cases, the number of combined soil tests is substantially less than the 100 soil tests that would be required if each sample were tested individually. The choice of sampling patterns and sample combination plan may be determined in advance using an algorithm, as discussed further later in this disclosure.

The samples may be collected from contiguous locations or non-contiguous locations across the area. Furthermore, while this application discusses the sampling pattern as being a grid, with combination samples created from the rows and columns, this is for ease of explanation only. The sampling pattern is not limited to grids but rather any pattern may be used. Likewise the combination samples are not limited to combining samples from a row or column but rather any pattern of combination that provides sufficient useful data may be used.

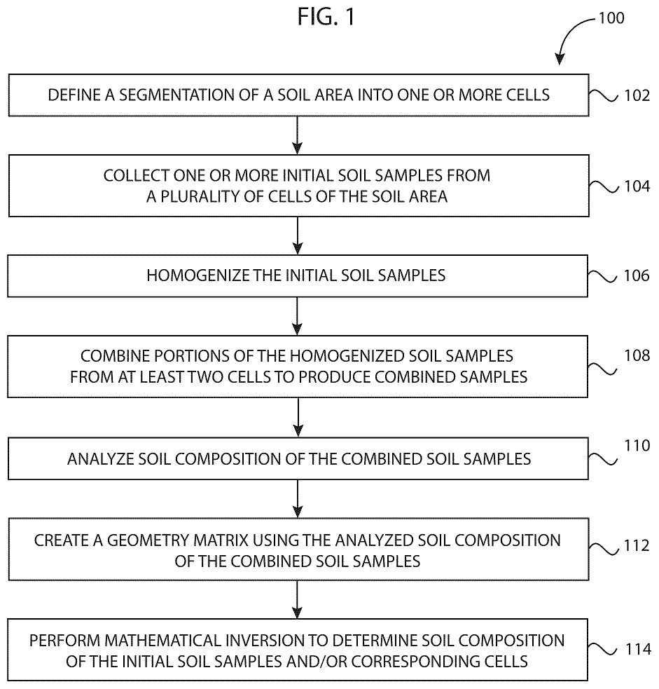

FIG. 1 shows a method 100 for soil composition analysis, according to an embodiment. At 102, a segmentation of a soil area into one or more cells is defined. The soil area is segmented into a plan including multiple cells, which cover geographic areas that may be of any size or shape. Typically, in agriculture, cells (also referred to as grids) about 2.5 acres and 5 acres are used. For high resolution purposes, cells of about 1 acre can be used; for efficiency, larger grids such as 7, 10 or 15 acres can be used as well. Depending on the purpose, cell size can be as little or as large as necessary.

At 104, one or more initial soil samples are collected from a plurality of the cells of the soil area. For example, one initial soil sample may be obtained from each of the plurality of cells.

At 106, the initial soil samples are homogenized. Homogenization of the soil sample includes steps to make the soil sample more uniform across the entire sample and may include mixing, blending, stirring and/or other similar steps geared towards equalizing the soil composition. It may further include steps such as drying, sieving, and/or combining with a liquid such as water. After homogenization, the sample may be approximately uniform in terms of the composition analysis that is being tested in this process.

At 108, portions of the homogenized soil samples from at least two cells are combined to produce a plurality of combined soil samples. The 108 includes using a portion of at least one homogenized soil sample from one cell in two or more independent combined soil samples. As such, the combining of the homogenized soil samples, 108, includes using a soil sample from one cell in two or more different combined soil samples. In some embodiments, a portion of each homogenized soil sample is combined with a portion of at least two different homogenized soil samples to form a plurality of combination soil samples, each combination soil sample including portions of homogenized soil samples from at least three cells. Each initial soil sample may be used in a plurality of combination soil samples such as two or more combination soil samples, and each combination soil sample may include soil from two or more homogenized soil samples. Furthermore, the plurality of combination soil samples may each include the same number of homogenized soil samples or may contain different numbers of homogenized soil samples. While step 108 and other parts of this disclosure refer to portions of an initial soil sample, it should be understood that the initial sample collected in step 104 may be homogenized and divided into portions used to make the combination samples. Alternatively, multiple initial soil samples may be obtained from a cell and these separate samples from the same cell may also be considered as portions of an initial soil sample.

At 110, soil composition of the combined soil samples is analyzed. Soil composition analysis may include analysis for one soil aspect or many soil characteristics, using one method or many methods to obtain the value of the result. While some testing may be performed in the field, oftentimes such testing may include transportation of the individual or combination soil samples to a testing facility such as a laboratory. For example, combination soil samples may be created in the field from the homogenized soil samples, and only these combination soil samples may then be delivered to a laboratory for analysis, with no separate testing of the initial soil samples used to make the combination samples.

Any of the steps 102-110 may be performed by a person, machine, robot, or a combination thereof. For example, collecting the samples, such as at the collection location or in proximity to the collection location such as the field, at the time of collection. Alternatively, one or both of steps 106 and 108 could be performed at a later time, by the same person, machine or robot that collected the samples or by a different person, machine or robot, for example. Any of the steps can be carried out in a close succession, simultaneously, or over a long period of time. For example, initial soil samples can be collected and immediately homogenized, while the mixing step could be carried out at a different time and/or location. Alternatively, the samples can be collected first, then transported to a different location for further processing. Yet in another embodiment, the process of soil collection, homogenization, and mixing can be automated, and some/each step can be carried out simultaneously. For example, the N-th sample is being collected, the N−1 is being homogenized after having been collected, the N−2 and N−3 are being combined after having been homogenized, etc.

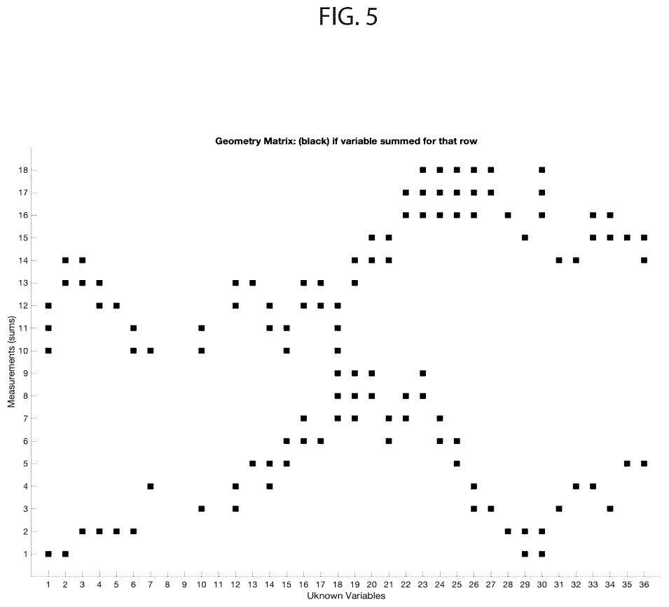

At 112, a geometry matrix is created using the analyzed soil composition of the combined soil samples. The geometry matrix (FIG. 5) is a two-dimensional matrix that relates the known measurements (M sums) to the unknown values to be estimated (N core carbon percent). For the example case under study here, there are 18 summations, and 36 carbon core samples to be estimated.

The geometry matrix is an extremely important part of the tomographic inversion process, and most tomographic problems in the real world have little control over the geometry matrix. However, for the problem under consideration here, we have complete control over both determining the total number of sums taken, and also what core samples are in each sum. Thus, one important piece of research is to determine what is the optimal geometry matrix for a given field, and the required set of unknown variables to be estimated.

At 114, mathematical inversion is performed to determine soil composition of the initial and/or homogenized soil samples, and/or the corresponding cells.

In an embodiment, defining a segmentation of a soil area into one or more cells can include defining a 3-dimensional segmentation of the soil area (volume). In some embodiments, the segmentation can include segmentation of the soil area in a planned set of 2-dimensional cells, which may be at the surface or at a defined depth below the surface. For example, the soil can be collected within a predetermined depth range, such as a depth between about 1 inch and about 48 inches below the soil surface, or between about 3 inches and about 24 inches below the soil surface, or between about 6 inches and about 18 inches below the soil surface, etc.

In some embodiments, collecting one or more initial soil samples, 104, can include collecting one or more samples from every cell of the segmented soil area; in other embodiments, collecting initial soil samples, 104, can include collecting one or more samples from less than every cell, such as only from a selection of one or more cells of the segmented soil area. The cells from which soil is collected may be adjacent to or separated from each other.

In various embodiments, segmenting the soil area can include segmenting the soil area into 2 or 3-dimensional grid cells. The grid may include one or more rows by one or more columns, optionally by one or more layers across a soil area. Alternatively, the grid may include any other division of the area which may or may not include rows or columns. The combining of the initial soil samples can include combining the samples along the one or more rows and/or one or more columns and/or one or more diagonals, and/or one or more layers. Alternatively, other combinations of soil samples may be performed unrelated to the columns or rows or other configuration of the cells. In some embodiments, the segmenting the soil area can include segmenting the soil area into 2-dimensional and/or 3-dimensional cells of variable shape and/or size. Segmenting may divide the entire soil area into cells, or only a portion or portions of the soil area.

For example, embodiments including a 2-dimensional segmentation may be represented by a grid of X rows (R1,R2, . . . RX) perpendicular to Y columns (C1,C2, . . . CY). The segmentation may be across a soil surface, or at a depth beneath the surface, or within a range of depths beneath the soil surface, for example. An initial soil sample may be obtained from one cell and may be used in two or more independent combined soil samples, 106. For example, the initial samples across one row, R1, (R1C1+R1C2+ . . . +R1CY) may be combined to create a first combined soil sample (Scom1), and the initial soil samples across one column, C1, (R1C1+R2C1+ . . . +RXC1) may be combined to create a second combined soil sample (Scom2), wherein the column C1 intersects with the row R1. As a result, one initial sample, R1C1, is included in both (Scom1 and Scom2) combined soil samples. However, while this example describes the cells as arranged in rows and columns, this is for ease of understanding, and the cells may have any formation and the samples may be combined in any manner that allows for mathematical inversion to predict their results.

For example, at 108, using at least one initial soil sample from one cell in two or more independent combined soil samples can include, but is not limited to, any of the following approaches. One sample can be collected from each cell, and then split into two or more parts, each part to be combined with parts of other initial samples from other cells processed in the same manner. Another approach involves collecting two or more samples for each cell, and using one or more whole (undivided) samples to combine with other whole (undivided) samples from other cells to create combined soil samples. Either way, the soil from one cell must be included in at least two combined samples. In some embodiments, the soil from each cell is included in at least two combined samples.

In various embodiments, analyzing soil composition, 110, can include analyzing physical, chemical, and/or biological aspects of the soil sample. For example, analyzing soil composition 110 can include analyzing or measuring soil texture (such as sand, silt, loam, and/or clay content), soil compaction, gas and/or moisture content, rocks and mineral particles, contaminants (such as pesticides, petroleum products, radon, asbestos, lead, chromated copper arsenate, and creosote), and/or nutrients (such as carbon, hydrogen, nitrogen, oxygen, phosphorus, potassium, calcium, magnesium, and sulfur) levels. Additionally or alternatively, analyzing soil composition 110 can include analyzing organic matter and/or microbial biomass (such as bacteria, fungi, and protozoa) levels. Analyzing soil composition 110 can also include analyzing the pH of soil and/or the chemical composition (including such elements as potassium, chlorine, iron, boron, manganese, zinc, copper, nickel, molybdenum, and/or heavy metals, for example).

In an embodiment, performing mathematical inversion, 114, can include performing tomographic inversion. Additionally or alternatively, performing mathematical inversion, 114, can include applying one or more 3D VAR techniques. Additionally or alternatively, performing mathematical inversion, 114, can include applying one or more of the following: basis function expansion, Tikhonov regularization, and/or singular value decomposition, or other methods to determine the predicted or estimated values of the initial soil samples from the measured values of the combination samples. In an embodiment, performing tomographic inversion, 114, can include producing inverted cell values. Additionally, the inverted cell values can be used to produce an interpolated map. Afterwards, the Kriging method or any other interpolation method or a combination of such methods can be applied.

In some embodiments, the method 100 may further include using the determined soil composition of the initial and/or homogenized soil samples and/or the corresponding cells to estimate soil composition of the soil area. For example, interpolation can be performed using the Kriging method or other appropriate methods.

In some embodiments, the method 100 may further include applying machine learning to the determined soil composition for soil modeling. Machine learning may be applied before the inversion process, such as to help constrain the solution. Additionally or alternatively, after the inversion process, a machine learning model may be applied to improve the resolution. Additionally or alternatively, the method 100 can include predicting future soil composition of the area from which the initial samples were gathered. For example, machine learning may use the determined values along with other data such as past values or other data to predict future values of the analyzed soil characteristic. Similarly, machine learning may use determined values from the past, such as from one or more previous years or seasons, along with other data such as topography, weather patterns, historical crop growth, historical soil treatment, and/or sensor data, to predict current values of the analyzed soil characteristic.

In various embodiments, the method 100 may further include applying proximal and/or remote sensing data to the inversion process. The remote data (such as satellite imagery) and/or proximal sensing data (such as EMI, GPR, NDVI, LIBS, INS, gamma, optical-spectrographic sensors, etc.) can be used to improve, inform and/or constrain the subsequent inversion process. This data may be collected simultaneously with the initial soil sample collection, or may be previously collected or may be collected at a later time.

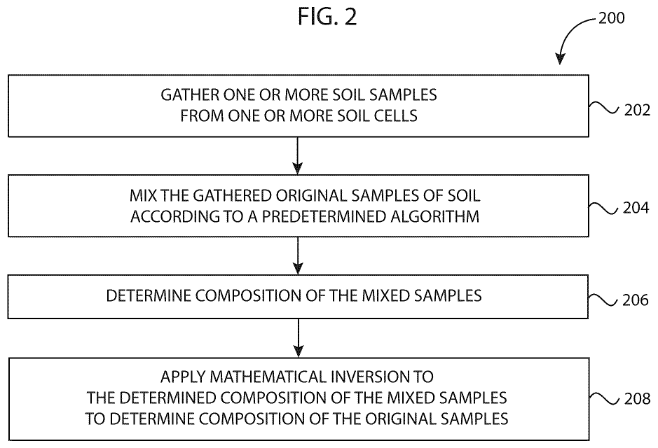

FIG. 2 shows a method 200 for processing soil samples, according to various embodiments. At 202, one or more samples of soil are gathered from one or more soil cells. For example, for the simplest case of a 2×3 matrix, five samples may be necessary for the analysis in order to recover all six locations. For the 3×3 matrix, from only six samples all nine locations can be recovered. The method 200 may further include using a sampling robot, especially for larger collection plans. In other embodiments, several samples may be collected from each location/cell. At 204, the gathered original samples of soil across the one or more cells are mixed according to a predetermined algorithm to produce a plurality of mixed samples, wherein each original sample is represented in at least two distinct mixed samples. At 206, composition of the mixed samples is determined. At 208, mathematical inversion is applied to the determined composition of the mixed samples to determine composition of the one or more soil cells.

In some embodiments, gathering one or more samples of soil, 202, can include can include gathering one or more original samples of soil from a segmentation of one or more rows of soil cells by one or more columns of soil cells across a soil area, and combining original samples of soil along the one or more rows, and/or along the one or more columns. Depending on an embodiment, the soil cells can be adjacent to each other, or can be separated from each other by some soil that is not being sampled or included as cells or considered in the analysis.

In an embodiment, the method 200 can further include homogenizing the gathered original soil samples before mixing the original samples.

In some embodiments, determining composition of the mixed samples, 208, can include analyzing physical, chemical and/or biological aspects of the soil area. For example, the method 200 can include analyzing or measuring soil texture (such as sand, silt, loam, and/or clay composition), soil compaction, gas and/or moisture content, rocks and mineral particles, contaminants (such as pesticides, petroleum products, radon, asbestos, lead, chromated copper arsenate, and/or creosote), and/or nutrients (such as carbon, hydrogen, nitrogen, oxygen, phosphorus, potassium, calcium, magnesium, and/or sulfur) levels. Additionally or alternatively, the method 200 can include analyzing organic matter, and/or microbial biomass (such as bacteria, fungi, and/or protozoa) composition. Determining the composition of the mixed samples can also include analyzing pH of soil and/or chemical composition (including such elements as potassium, chlorine, iron, boron, manganese, zinc, copper, nickel, molybdenum, and/or heavy metals, for example).

In an embodiment, applying mathematical inversion, 208, can include performing tomographic inversion. Additionally or alternatively, 208 can include applying one or more 3D VAR techniques. In some embodiments, applying tomographic inversion, 208, may include applying one or more methods such as 3DVAR, basis function expansion, Tikhonov regularization, and/or singular value decomposition, or other methods to determine the predicted or estimated values of the initial soil samples from the measured values of the combination samples. In some embodiments, the method 200 may further include producing an interpolated map of the soil area containing the soil cells.

In some embodiments, the method 200 may further include using the determined composition of the original samples in machine learning for soil composition modeling. In an embodiment, the machine learning may be applied before the inversion process, such as to help constrain the solution. Additionally or alternatively, after the inversion process the machine learning model may be applied to improve the resolution. Additionally or alternatively, the method 200 may include predicting future soil composition of the soil area from which the original samples were gathered.

In some embodiments, the method 200 method may further include applying proximal and/or remote sensing data to the inversion process. The remote data (such as satellite or remote sensor) and/or proximate sensing data (such as EMI, GPR, NDVI, LIBS, INS, gamma, optical-spectrographic sensors, etc.) can be used to improve, inform and/or constrain the subsequent inversion process.

The soil segmentation, sampling plan, and sample combination plan used in various embodiments may be determined in advance of beginning sample collection using a predetermined algorithm, ensuring that every sample location is represented in at least two composite samples.

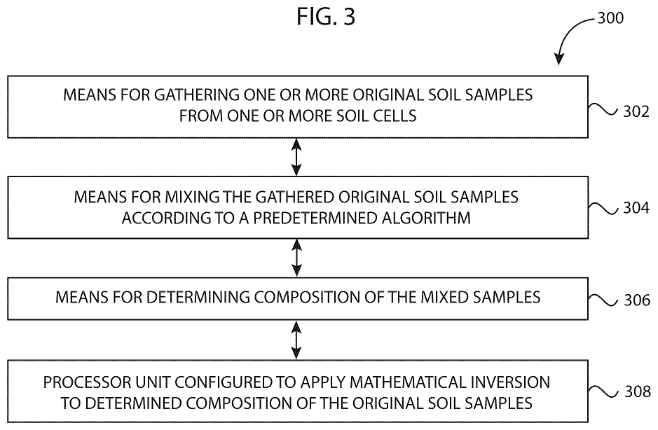

FIG. 3 shows a system 300 for soil composition analysis, according to an embodiment. The system 300 includes 302, a means for gathering one or more original samples of soil from one or more soil cells; 304, a means for mixing the gathered original samples of soil according to a predetermined algorithm; 306, a means for determining composition of the mixed samples; and 308, a processor unit configured to apply mathematical inversion to the determined composition of the mixed samples to determine composition of the original samples.

In an embodiment, the processor unit, 308, can be further configured to correlate proximal and/or remote sensing data with the output of the mathematical inversion. Additionally or alternatively, the processor unit, 308, can be further configured to generate an interpolated map of the soil cells.

The method 300 may include using means of gathering of the original soil samples. Examples of means for gathering one or more original samples of soil include manual and automated mechanisms, including robotic processes, using soil sampling tools such as probes, corers, shovels, scoops, or other tools utilizable for extraction of soil. One specific example of a tool which may be used is an AMS ⅞″ diameter 30 cm (12 inch) length soil sample probe, though probes with different diameters and/or lengths could alternatively be used.

Examples of means for mixing portions of the gathered homogenized original samples of soil include tools for mixing the combined soil samples in a container, which may be the same or different from the container used to collect the original sample, to make the combined samples uniform, such as a stirring paddle, rod, whisk or other mixing or blending tool in a container such as a bucket, bowl, or other container, and/or other mixing devices which may mix the soil samples such as agitators or shakers. The mixing may be performed manually or may be fully or partially automated and performed by a machine or robot, for example.

The means for homogenizing the gathered samples may be used in combination with sieves in some embodiments. The samples may be homogenized in dry form or may be combined with a liquid such as water (for example, pure water, distilled or reverse osmosis water). The means for homogenizing may, therefore, include a water tank and dispenser for combining the original soil sample (or a portion of the original soil sample) with a quantity of water, such as a predetermined metered quantity, and mixing or blending the water and soil in a mixer or blender until homogenized. The means for homogenizing may further include a weight measuring device such as a scale and/or volume measuring device to determine the quantity of soil in the sample being homogenized for mixing with an appropriate amount of water.

The means for gathering the one or more original samples, the means for homogenizing the one or more samples, and the means for mixing the gathered homogenized original samples may all be included in a single machine such as an independently operating mobile machine like a robot. The mobile robot may include wheels and a power source such as a battery or motor for moving across the soil area to collect the samples. It may include means for weight and/or volume measurement for measuring and controlling the amount of soil in the original sample, portions of the sample being homogenized, and/or portions of the homogenized sample being combined. It may include a tank of liquid, such as distilled water or reverse osmosis water, for use in homogenization of the sample. It may further include one or more processors, such as central processing units or multicore processing units containing the logic circuitry of the robot, to receive instructions regarding the sampling locations and combining of the samples according to a predetermined algorithm, and for carrying out those instructions. The robot may further include GPS detectors for navigating a positioning itself at the sample locations. The robot may seal the mixed samples in containers and deliver them to a location or a user when complete.

Examples of means for determining the composition of the mixed samples include laboratory equipment and processes such as processes using chemistry such as wet chemistry, loss on combustion testing, and/or other soil testing methods. Specific testing methods which may be used include wet chemistry tests like Bray and Mehlich, specialized equipment like NIR (Near Infrared Spectroscopy), X-Ray spectroscopy, chromatography, and other methods and equipment. A loss on ignition test may be used to measure organic carbon, for example. Means for performing a high throughput optical Polymerase Chain Reaction (PCR) may also be used.

In some embodiments, a processor unit such as a central processing unit or a multicore processing unit that includes the logic circuitry and programming for performing the mathematical inversion, may be a part of a computer or server, and may be further configured to correlate proximal and/or remote sensing data with the output of the mathematical inversion. Additionally or alternatively, the processor unit may be further configured to generate an interpolated map of the soil cells.

### Example

Soil samples were obtained from a field. A grid plan was used for segmenting the field and taking soil samples as shown in FIG. 5. The area of the field 500 was “divided” into a 2-dimensional grid 502 having 9 segments in a 3 by 3 configuration as indicated by the dashed lines. The resulting grid had 3 columns and 3 rows. One soil sample was obtained from each segment, as indicated by the “x.” The samples are arbitrarily numbered x1-x9. Carbon was measured in each of these samples, and these results represent the true value of carbon at each sample location. Carbon levels were also estimated using the methods of the invention as described below. These calculated or determined carbon levels were then compared to the true measured values to assess the accuracy of the method.

Samples x1-x9 were split into multiple portions for combination with other samples as follows. For each of the three rows, a portion of each sample was combined across the row to form a new combination sample for that row. Likewise, for each of the three columns, a portion of each sample in the column was combined to form a new combination sample for that column. The result was 6 combination samples produced from 9 original samples. The composition of the combination samples is shown in table 1, below.

TABLE 1

| Portions of | |
|---|---|
| individual samples | Combined Sample |
| x1 + x2 + x3 | row 1 combination |
| x4 + x5 + x6 | row 2 combination |
| x7 + x8 + x9 | row 3 combination |
| x1 + x4 + x7 | column 1 combination |
| x2 + x5 + x8 | column 2 combination |
| x3 + x6 + x9 | column 3 combination |

The combination samples were mixed sufficiently to combine the sample portions included in the combination sample and make the composition of the combination samples uniform. After mixing, the carbon composition of each of the combination samples was measured. The carbon levels found in each combination sample were then used to mathematically predict the carbon levels at each sample location as described further below.

The combination sample results were analyzed using a 3D variation method, also sometimes referred to as a maximum likelihood variation method or a Kalman filter method. The algorithm used for performing this analysis was a data assimilation program known as Ionospheric Data Assimilation Four Dimensional (IDA4d). This method regularizes solutions and adds constraints to an underdetermined problem by using probabilistic correlation functions, as a function of space, of the variant of interest which in this example is carbon. It further uses similar correlation functions between the measurements and the variable of interest and uses similar correlation functions for multivariate cases between variables. An initial guess or forecast for the variable of interest was used at each sample location.

The following solution equation was used to determine the estimated amount of carbon in each sample based on the measurements of the combination samples:

$${\vec{x}}_{a} = {\vec{x}}_{f} + {\tilde{P}}_{a} {\tilde{H}}^{T} {\tilde{W}}^{- 1} ( \vec{d} - \tilde{H} {\vec{x}}_{f} )$$

in which \tilde{P}ₐ=[\tilde{H}^{T}\tilde{W}⁻¹\tilde{H}+\tilde{P}_{f}⁻¹]⁻¹. The vector \vec{d} is the data. In this case, it was the percent of sums of carbon cores. The vector \vec{x}_{f} is a forecast, or initial guess of what the desired solution estimation is. \tilde{H} is the “geometry matrix”, or connection matrix that connects the carbon cores to the measured sums. \tilde{W} is a matrix (normally diagonal) of the data/observation error covariances. In this example, the inverse of this was used. \tilde{P}ₐ is the analyzed error covariance. That is the estimated posterior covariance error of the solution. \tilde{P}_{f} is the forecast (guess, model) error covariance. \vec{x}ₐ is the analyzed solution, that is the estimated solution of the variable desired, which in this example was carbon in this case.

In this example, the implementation is general. In other examples, the vector x could consist of multiple variables, as well as observations and derived parameters (such as conductivity), for example, that are all estimated on a grid. In addition, the regularization and constraints were implemented by a forecast/guess initial solution and the forecast covariance, as described further below.

The error covariance was made up of the variance of the variables and the spatial correlations between them. For two variables at difference spatial points, the spatial correlation is the correlation between the two variables. The spatial dependence was taken as depending only on the distance between the two variables, and not the direction or specific region of the field the Y were on. For carbon, the spatial dependent was ρ_{cc}(Δr)=<(Δr)ᵢⱼ>. where cc means carbon-carbon correlation, (Δr)ᵢⱼ means the separation distance between a carbon measurement at I and one at j, and the brackets mean average over all ij pairs. Note that the measurement does not have to be a measurement of carbon itself. An EMI quadrature 63030 Hz (q64) with carbon could be used, such that ρ_{cq64}(Δr)=<(Δr)ᵢⱼ> where now the (Δr)ᵢⱼ is the distance between a q64 measurement and carbon.

The data was analyzed using 4 different alternative approaches as follows. The initial guess was set at either zero or 1. For each of these, either all 6 combination samples were used or only 4 combination samples for the determination. When only 4 combination samples were used, the combination samples were only those taken from the outer rows and columns, and the combination samples from the middle row and middle column were not used for the determination.

Average error was calculated as <truth−estimate>. Average absolute error was calculated as <|truth-estimate|>. Average percent error was calculated as <(truth−estimate)/truth>. Average absolute percent error was calculated as <(|truth−estimate|)/|truth|>. Skill was calculated as 1−rms(truth−estimate)/rms(truth−guess). As such, skill compares the calculated estimate to the initial guess, with 1 indicating the calculated estimate was perfect, 0 indicating that the estimate was no better than the initial guess, and a number less than zero indicating the calculated estimate was worse than the guess.

The results of the determinations, using the four different approaches, are shown in Table 2, below.

TABLE 2

| | guess = 1, | guess = 1, | guess = 0, | guess = 0, |
|---|---|---|---|---|
| Error | 6 comb. samples | 4 comb. samples | 6 comb. samples | 4 comb. samples |
| Average error | .052 | .053 | .067 | 0.13 |
| Ave. abs. | 0.118 | 0.107 | 0.14 | 0.18 |
| error | | | | |
| Ave. % error | 3.7% | 3.7% | 4.9% | 11.2% |
| Ave. abs. % | 11.2% | 10% | 13.5% | 16.9% |
| error | | | | |
| Skill | 0.23% | 2.96% | 84% | 79% |

As shown above, for an initial guess of 1, the skill results suggest that there was not much improvement of the determined results over the initial guess. In addition, the determined results that were obtained using only 4 combination samples were as good as the determined results using 6 combination samples. When the initial guess was zero, the skill score suggests that there was a large improvement in the determined results as compared to the initial guess for both the 6 combination sample results and the 4 combination sample results.

The data demonstrate that a better initial guess (such as a guess of 1) results in a better estimate, even when there is less data. However, even when a poor initial guess is used (such as a guess of 0), the method still converges to a very good estimate which is close to the estimate obtained with a good initial guess, even when less data is used (4 combination samples instead of 6). Therefore, the solution is not especially sensitive to the guess. Also, even when less data is used, a good sampling procedure still produces good results.

As used herein, terms “segmentation” and “grid” shall be considered synonymous, and refer to 2-dimensional and/or 3-dimensional objects unless further definition is provided. As used herein, terms “cell” and “voxel” shall be considered synonymous, and refer to 2-dimensional and/or 3-dimensional objects unless further definition is provided. As used herein, terms “area” and “volume” shall be considered synonymous, and refer to 2-dimensional and/or 3-dimensional objects, unless further definition is provided. A device or structure that is “configured” in a certain way is configured in at least that way, but may also be configured in ways that are not listed.

Device and method components are meant to show only those specific details that are pertinent to understanding the embodiments of the present disclosure so as not to obscure the disclosure with details that will be readily apparent to those of ordinary skill in the art having the benefit of the description herein. In various embodiments, the sequence in which the elements appear in exemplary embodiments disclosed herein may vary. Two or more method steps may be performed simultaneously or in a different order than the sequence in which the elements appear in the exemplary embodiments.

In the specification and/or figures, typical embodiments of the invention have been disclosed. The present invention is not limited to such exemplary embodiments. The use of the term “and/or” includes any and all combinations of one or more of the associated listed items. The figures are schematic representations and so are not necessarily drawn to scale. Unless otherwise noted, specific terms have been used in a generic and descriptive sense and not for purposes of limitation.

1. A method for soil composition analysis, comprising:
defining a segmentation of a soil area into one or more cells;
collecting one or more initial soil samples from a plurality of the cells of the soil area;
homogenizing the initial soil samples;
combining portions of the homogenized soil samples from at least two cells to produce a plurality of combined soil samples;
analyzing soil composition of the combined soil samples;
creating a geometry matrix using the analyzed soil composition of the combined soil samples; and
performing mathematical inversion to determine soil composition of the initial and/or homogenized soil samples, and/or the corresponding cells;
wherein the combining portions of the homogenized initial soil samples comprises using a portion of at least one homogenized soil sample from one cell in two or more independent combined soil samples.

2. The method according to claim 1, wherein defining a segmentation of a soil area into one or more cells includes defining a 3-dimensional segmentation.

3. The method according to claim 1, wherein analyzing soil composition includes analyzing physical, chemical, and/or biological aspects of the soil area.

4. The method according to claim 1, herein performing mathematical inversion includes performing tomographic inversion.

5. The method according to claim 1, wherein performing tomographic inversion includes applying a 3D VAR technique.

6. The method according to claim 1, further comprising using the determined soil composition of the initial and/or homogenized soil samples and/or the corresponding cells to estimate soil composition of the soil area.

7. The method according to claim 1, further comprising using previously determined soil composition of the soil area to determine accuracy of the determined soil composition of one or more of the initial and/or homogenized soil samples and/or the corresponding cells.

8. The method according to claim 1, further comprising applying machine learning to the determined soil composition for soil modeling.

9. A method for processing soil samples, comprising:
gathering one or more samples of soil from one or more soil cells;
mixing the gathered original samples of soil across the one or more cells according to a predetermined algorithm to produce a plurality of mixed samples, wherein each original sample is represented in at least two distinct mixed samples;
determining composition of the mixed samples; and
applying mathematical inversion to the determined composition of the mixed samples to determine composition of the one or more soil cells.

10. The method according to claim 9, wherein gathering one or more samples of soil includes gathering one or more original samples of soil from a segmentation of one or more rows of soil cells by one or more columns of soil cells across a soil area, and combining original samples of soil along the one or more rows, and/or along the one or more columns.

11. The method according to claim 9, further comprising homogenizing the gathered original soil samples before mixing the original samples.

12. The method according to claim 9, wherein determining composition of the mixed samples includes analyzing physical, chemical and/or biological aspects of the soil area.

13. The method according to claim 9, wherein applying mathematical inversion includes performing tomographic inversion.

14. The method according to claim 9, wherein applying topographic inversion includes applying a 3D VAR technique.

15. The method according to claim 9, further comprising producing an interpolated map of the soil cells.

16. The method according to claim 9, further comprising using the determined composition of the original samples in machine learning for soil composition modeling.

17. The method according to claim 9, further comprising applying proximal and/or remote sensing data to determine composition of the one or more soil cells.

18. A system for soil composition analysis, comprising:
a means for gathering one or more original samples of soil from one or more soil cells;
a means for mixing the gathered original samples of soil according to a predetermined algorithm;
a means for determining composition of the mixed samples; and
a processor unit configured to apply mathematical inversion to the determined composition of the mixed samples to determine composition of the original samples.

19. The system according to claim 18, wherein the processor unit is further configured to correlate proximal and/or remote sensing data with the output of the mathematical inversion.

20. The system according to claim 18, wherein the processor unit is further configured to generate an interpolated map of the soil cells.
